(19) United States
(12) Patent Application Publication
(10) Pub. No.: US 2004/0001844 A1
(43) Pub. Date: Jan. 1, 2004

(54) Lewis Y epitope-containing mucin fusion polypeptide vaccines, compositions and methods of use thereof

(76) Inventor: Jan Holgersson, Huddinge (SE)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/421,198

(22) Filed: Apr. 22, 2003

Related U.S. Application Data
(60) Provisional application No. 60/375,100, filed on Apr. 22, 2002.

(51) Int. Cl. A61K 39/00; C07K 14/47; A61K 31/739

(52) U.S. Cl. 424/185.1; 530/395; 514/54

(57) ABSTRACT

The present invention provides compositions and methods for cancer vaccine immunogenicity using lewis Y mucin-immunoglobulin fusion proteins.

### RELATED APPLICATIONS

This application claims priority to U.S. Ser. No. 60/375,100, filed Apr. 22, 2002, the contents of which is incorporated herein by reference in its entirety.

### FIELD OF THE INVENTION

The invention relates to compositions and methods of protein vaccines and their use in preventing and treating diseases such as cancer.

### BACKGROUND OF THE INVENTION

Cancer vaccines using whole cell lysates derived from autologous or allogeneic tumors are currently used to target well-characterized tumor-associated antigens. These tumor-associated antigens are often proteins or glycoproteins, but can also be carbohydrate moieties. Carbohydrates are among the most well-studied targets for anti-bacterial vaccine therapy. Immune responses against carbohydrate moieties are mainly antibody-dependent, and antibodies against capsular polysaccharides of several bacteria have been shown to protect from subsequent bacterial challenge. Similarly, antibodies directed at tumor-associated antigens may be able to reduce or eliminate circulating tumor cells and micrometastases. Non-human animal models and clinical trials have shown that antibodies direced at tumor-associated antigens are able to mediate protection from tumor recurrence. However, carbohydrates are poor immunogens and are generally unable to be recognized by the T cells

Mucins such as MUC1, and mucin-like molecules with highly O-glycosylated domains, such as P-selectin glycoprotein ligand-1 (PSGL-1), are extensively glycosylated high molecular weight (>200 kD) proteins, and are targets for numerous glycosylating enzymes, including α1,2 fucosyltransferase, α1,3 fucosyltransferase, and α1,3 galactosyltransferase. Mucins are abundantly expressed in normal cells such as leukocytes and in many human cancers of epithelial origin.

### SUMMARY OF THE INVENTION

The invention is based in part in the discovery that fusion proteins containing Lewis Y (Le^{y}) carbohydrate epitopes increase vaccine immunogenicity. Mucins, which are targets for the fucosyltransferases that generate Lewis Y epitopes, are particularly useful in vaccines.

The invention features a purified tumor vaccine including a polypeptide that is glycosylated by a α1,2 fucosyltransferase and α1,3 fucosyltransferase. The polypeptide contains multiple Le^{y} epitopes. For example, the polypeptide includes more Le^{y} epitopes epitopes than a wild-type P-selectin glycoprotein ligand-1 polypeptide.

In another aspect, the invention relates to a purified tumor vaccine including a first polypeptide including a tumor-associated carbohydrate epitope, operably linked to a second polypeptide including an immune response stimulator polypeptide. The first polypeptide that is glycosylated by an α1,2 fucosyltransferase and α1,3 fucosyltransferase. The tumor-associated carbohydrate epitope is for example Lewis Y, Lewis A, Lewis X, and Lewis B. The first polypeptide contains multiple tumor-associated carbohydrate epitopes. Alternatively, the first polypeptide contains more tumor-associated carbohydrate epitopes than a native (i.e, wild-type) polypeptide. A native polypeptide is a polypeptide that naturally expresses the tumor-associated carbohydrate epitopes.

In another aspect, the invention provides a purified tumor vaccine including an adjuvant polypeptide including a first polypeptide that is a mucin polypeptide and is glycosylated by a α1,2 fucosyltransferase (such as FUT1 or FUT2) and α1,3 fucosyltransferase (such as FUT3, FUT4, FUT5, FUT6, FUT7, or FUT9) operably linked to a second polypeptide that includes at least a region of an immunoglobulin polypeptide, and an immune response stimulator polypeptide. The adjuvant polypeptide is operably linked to the immune response stimulator polypeptide, such as by a covalent linkage.

The first polypeptide is a mucin polypeptide. The polypeptide is a monomer. Alternatively, the polypeptide is a dimer. The polypeptide is for example a P-selectin glycoprotein ligand-1 polypeptide. The polypeptide includes at least a region of a P-selectin glycoprotein ligand-1, such as the extracellular portion of a P-selectin glycoprotein ligand-1. In some embodiments, the first polypeptide includes multiple Le^{y} epitopes.

The immune response stimulator polypeptide is a T-cell stimulator polypeptide. The T-cell stimulator polypetide is keyhole limpet hemocyanin, a heat shock protein (HSP) such as HSP60 or HSP70, or or a superantigen such as Staphylococcus enterotoxin.

The purified tumor vaccine of the invention also includes an immunoglobulin polypeptide. This immunoglobulin polypeptide includes a region of a heavy chain immunoglobulin polypeptide. Alternatively, the immunoglobulin polypeptide includes an Fc region of an immunoglobulin heavy chain.

The invention also provides nucleic acids encoding the tumor vaccines, vectors containing these nucleic acids, and cells including these vectors.

The present invention further provides a method of immunizing a subject, e.g., a mammal, by administering to the subject a tumor vaccine according to the invention.

The present invention additionally provides a method of preventing or alleviating a symptom of cancer in a subject, by administering to the subject a tumor vaccine according to the invention. The cancer is for example abreast, lung, colon, prostate, pancreas, cervix or skin cancer.

The invention further features methods of increasing immune cell activation by contacting a cell with a vaccine according to the invention. The cell is a B cell or a T-cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention is based in part on the discovery that mucin fusion proteins having Lewis Y (Le^{y}) epitopes are effective cancer vaccines.

Several carbohydrate-based, tumor-associated antigens have been identified in melanomas, neuroblastomas, sarcomas, small cell lung cancer, and in cancers of the breast, prostate, lung, colon, ovary and stomach. In order to generate high-titered antibody responses to tumor-associated carbohydrate moieties, the present invention provides a polyvalent conjugate vaccine having potent adjuvant properties. Recombinant mucins are designed to carry multivalent expression of tumor-associated carbohydrate epitopes, such as the Le^{y} epitope, together with peptide sequences which bind MHC class II molecules and elicit T helper lymphocytes. The lewis Y epitope density facilitates recognition by specific T cells are induced. T cells are attracted independently by coupling the mucin-Le^{y} complex to proteins (e.g.,) that facilitate the formation of a bridge between the MHC class II molecule and the T cell receptor, creating a pseudo-cognate interaction between T cells and B cells.

As used herein, the following definitions are supplied in order to facilitate the understanding of this case. To the extent that the definitions vary from meanings known to those skilled in the art, the definitions below control.

By “mucin” is meant any polypeptide with one or more O-glycosylated domains, that are targets for fucosyltransferases, such as α1,2 fucosyltransferase and α1,3 fucosyltransferase, as well as for the α1,3 galactosyltransferase.

By “biological component” is meant any compound created by or associated with a cell, tissue, bacteria, virus, or other biological entity, including peptides, proteins, lipids, carbohydrates, hormones, or combinations thereof.

By “adjuvant compound” is meant any compound that increases an immunogenic response or the immunogenicity of an antigen or vaccine.

By “antigen” is meant any compound capable of inducing an immunogenic response.

By “immunoglobulin” is meant a any polypeptide or protein complex that is secreted by plasma cells and that functions as an antibody in the immune response by binding with a specific antigen. Immunoglobulins as used herein include IgA, IgD, IgE, IgG, and IgM. Regions of immunoglobulins include the Fc region and the Fab region, as well as the heavy chain or light chain immunoglobulins.

By “antigen presentation” is meant the expression of an antigen on the surface of a cell in association with one or more major hisocompatability complex class I or class II molecules. Antigen presentation is measured by methods known in the art. For example, antigen presentation is measured using an in vitro cellular assay as described in Gillis, et al., J. Immunol. 120: 2027 1978.

By “immunogenicity” is meant the ability of a substance to stimulate an immune response. Immunogenicity is measured, for example, by determining the presence of antibodies specific for the substance. The presence of antibodies is detected by methods know in the art, for example, an ELISA assay.

By “immune response” or “immunogenic response” is meant a cellular activity induced by an antigen, such as production of antibodies or presentation of antigens or antigen fragments.

By “proteolytic degradation” is meant degradation of the polypeptide by hydrolysis of the peptide bonds. No particular length is implied by the term “peptide.” Proteolytic degradation is measured, for example, using gel electrophoresis.

The “cell” includes any cell capable of antigen presentation. For example, the cell is a somatic cell, a B-cell, a macrophage or a dendritic cell.

The invention provides mucin-immune response stimulator fusion proteins (refered to herein as “mucin-IRS fusion proteins”) containing a mucin polypeptide and an immune response stimulator polypeptide that are useful as vaccines. The vaccines are useful in methods of immunization an treating cancer in a subject. The fusion protein of the invention carries a tumor-associated carbohydrate epitope. For example, the fusion polypeptide carries the Le^{y}, Le^{x}, Le^{a}, or Le^{b} epitope. Preferably, the fusion protein carries the Le^{y} epitope. Alternatively the fusion protein carries teo or more tumor-associated carbohydrate epitopes.

Fusion Proteins

In various aspects the invention provides fusion protein includes a first polypeptide containing at least a portion of a glycoprotein, e.g., a mucin polypeptide operatively linked to a second polypeptide. By “at least a portion” is meant that the mucin polypeptide contains at least one mucin domain (e.g., an O-linked glycosylation site). Preferably, the mucin polypeptide glycosylated by an α1,2 fucosyltransferase, an α1,3 fucosyltransferase, or both an α1,2- and an α1,3 fucosyltransferase. As used herein, a “fusion protein” or “chimeric protein” includes at least a portion of a mucin polypeptide operatively linked to a non-mucin polypeptide. A “mucin polypeptide” refers to a polypeptide having a mucin domain. The mucin polypeptide has one, two, three, five, ten, twenty or more mucin domains. The mucin polypeptide is any glycoprotein characterized by a amino acid sequence substituted with O-glycans. For example, a mucin polypeptide has every second or third amino acid being a serine or threonine. The mucin polypeptide is a secreted protein. Alternatively, the mucin polypeptide is a cell surface protein.

Mucin domains are rich in the amino acids threonine, serine and proline, where the oligosaccharides are linked via N-acetylgalactosamine to the hydroxy amino acids (O-glycans). A mucin domain comprises or alternatively consists of an O-linked glycosylation site. A mucin domain has 1, 2, 3, 5, 10, 20, 50, 100 or more O-linked glycosylation sites. Alternatively, the mucin domain comprises or alternatively consists of a N-linked glycosylation site. A mucin polypeptide has 50%, 60%, 80%, 90%, 95% or 100% of its mass due to the glycan. Whereas a “non-mucin polypeptide” refers to a polypeptide of which at least less than 40% of its mass is due to glycans. A mucin polypeptide is any polypeptide encode for by a MUC genes (i.e., MuC1, MUC2, MUC3, MUC4, MUC5a, MUC5b, MUC5c, MUC6, MUC11, MUC12,etc.). Alternatively, a mucin polypeptide is P-selectin glycoprotein ligand 1 ( PSGL-1), CD34, CD43, CD45, CD96, GlyCAM-1, MAdCAM, red blood cell glycophorins, glycocalicin, glycophorin, sialophorin, leukosialin, LDL-R, ZP3, and epiglycanin. Preferably, the mucin is PSGL-1.

The mucin polypeptide contains all or a portion of the mucin protein. Alternatively, the mucin protein includes the extracellular portion of the polypeptide. For example, the mucin polypeptide includes the extracellular portion of PSGL-1 (e.g., amino acids 19-319 disclosed in GenBank Accession No. A57468). The mucin polypeptide also includes the signal sequence portion of PSGL-1 (e.g., amino acids 1-18), the transmembrane domain (e.g., amino acids 320-343), and the cytoplamic domain (e.g., amino acids 344-412).

The first polypeptide is generally glycosylated by an α1,2 fucosyltransferase and an α1,3 fucosyltransferase. In some aspects, the first polypeptide is also glycosylated by an α1,3 galactosyltransferase or an 1,3 N-acetylgalactosaminyltransferase, or another enzyme known to one of ordinary skill in the art to glycosylate a polypeptide. Suitable fucosyltransferase include for example, GenBank Accession Nos: NP_{—}002025, NM_{—}178032, NM_{—}031635 U17894, AB015634 and AF134414 and are incorporated herein by reference in their entirety.

Within the fusion protein, the term “operatively linked” is intended to indicate that the first and second polypeptides of the adjuvant polypeptide are chemically linked (most typically via a covalent bond such as a peptide bond) in a manner that allows for O-linked glycosylation of the first polypeptide. When used to refer to nucleic acids encoding a fusion polypeptide, the term operatively linked means that a nucleic acid encoding the mucin polypeptide and the non-mucin polypeptide of the adjuvant polypeptide are fused in-frame to each other. The non-mucin polypeptide is fused to the N-terminus or C-terminus of the mucin polypeptide, or to an internal amino acid of the mucin polypeptide. By “internal amino acid” is meant an amino acid that is not at the N-terminal or C-terminal of a polypeptide.

The non-mucin polypeptide is an immune response stimulator (IRS) polypeptide. By “immune response stimulator” is meant a compond that increases an immune response. The immune response stimulator polypeptide includes a T cell stimulator polypeptide. Exemplary T cell stimulator polypeptides include keyhole limpet hemocyanin (KLH), a heat shock protein (HSP), and a superantigen.

Keyhole limpet hemocyanin (KLH) is a copper-containing protein, isolated from the hemolymphs of the mollusk. KLH exists in five different aggregated states (in Tris buffer pH 7.4), which readily dissociate with moderated pH change. Subunit molecular mass is about 450 kDa.

Heat shock proteins (HSPs) include molecular chaperones that bind and stabilize proteins at intermediate stages of folding, assembly, translocation across membranes and degradation. HSP60 and HSP70 are preferred HSPs.

Superantigens include antigens that interact with a set of T lymphocytes, are not MHC class II restricted, and are able to interact with MHC class II molecules in an unprocessed form (generally, conventional antigens must be presented via a cell's endocytic pathway). Superantigens are capable of activating a diverse group of T cells. Superantigens include virally-encoded superantigens (e.g., murine mammary tumor virus' M1s; rabies virus nucleocapsid protein; Epstein-Barr Virus (EBV)-associated superantigen); pyrogenic toxin superantigens (PTSAgs) (e.g., Toxic Shock Syndrome Toxin-1 (TSST-1); staphylococcal enterotoxin A; staphylococcal enterotoxin B; and streptococcal scarlet fever toxin (SPEs); and other bacterial superantigens (e.g., staphylococcal exfoliative toxins; mycoplasma arthritidis mitogen; Yersinia enterocolitica and pseudotuberculosis superantigens, and streptococcal M protein). The murine M1s gene product is also a superantigen.

Alternatively, the fusion polypeptide is linked to an adjuvant polypeptide. An adjuvant polypeptide is a mucin polypeptide that is glycosylated by an α1,2 fucosyltransferase, and an α1,3 fucosyltransferase, and a second polypeptide that contains at least a region of an immunoglobulin polypeptide.

The vaccine is linked to one or more additional moieties. For example, the mucin-IRS protein may additionally be linked to a GST fusion protein in which the mucin-immune response stimulator fusion protein sequences are fused to the C-terminus of the GST (i.e., glutathione S-transferase) sequences. Such fusion proteins can facilitate the purification of mucin-IRS fusion protein. Alternatively, the mucin-IRS fusion protein may additionally be linked to a solid support. Various solid support are know to those skilled in the art. Such compositions can facilitate removal of anti-blood group antibodies. For example, the mucin-IRS protein is linked to a particle made of, e.g., metal compounds, silica, latex, polymeric material; a microtiter plate; nitrocellulose, or nylon or a combination thereof.

The fusion protein includes a heterologous signal sequence (i.e., a polypeptide sequence that is not present in a polypeptide encoded by a mucin nucleic acid) at its N-terminus. For example, the native mucin signal sequence is removed and replaced with a signal sequence from another protein. In certain host cells (e.g., mammalian host cells), expression and/or secretion of polypeptide is increased through use of a heterologous signal sequence.

A chimeric or fusion protein of the invention is produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene is synthesized by conventional techniques including automated DNA synthesizers. Alternatively, PCR amplification of gene fragments is carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fe region of an immunoglobulin heavy chain). A fusion protein-encoding nucleic acid is cloned into such an expression vector such that the fusion moiety is linked in-frame to the immunoglobulin protein.

Mucin-IRS fusion polypeptides may exist as oligomers, such as dimers, trimers or pentamers. Preferably, the mucin-IRS fusion polypeptide is a dimer. More preferably, the mucin-IRS fusion polypeptide is a dimeric PSGL-1 protein, or the extracellular region thereof.

The first polypeptide, and/or nucleic acids encoding the first polypeptide, is constructed using mucin encoding sequences are known in the art. Suitable sources for mucin polypeptides and nucleic acids encoding mucin polypeptides include GenBank Accession Nos. A57468, NP663625 and NM145650, CAD10625 and AJ417815, XP140694 and XM140694, XP006867 and XM006867 and NP00331777 and NM009151 respectively, and are incorporated herein by reference in their entirety.

The mucin polypeptide moiety is provided as a variant mucin polypeptide having a mutation in the naturally-occurring mucin sequence (wild type) that results in increased carbohydrate content (relative to the non-mutated sequence). For example, the variant mucin polypeptide comprised additional O-linked glycosylation sites compared to the wild-type mucin. Alternatively, the variant mucin polypeptide comprises an amino acid sequence mutations that results in an increased number of serine, threonine or proline residues as compared to a wild type mucin polypeptide. This increased carbohydrate content is assessed by determining the protein to carbohydrate ratio of the mucin by methods know to those skilled in the art.

The mucin polypeptide moiety is provided as a variant mucin polypeptide having mutations in the naturally-occurring mucin sequence (wild type) that results in a mucin sequence more resistant to proteolysis (relative to the non-mutated sequence).

The first polypeptide of fusion polypeptide includes full-length PSGL-1. Alternatively, the first polypeptide comprise less than full-length PSGL-1 polypeptide such as the extracellular portion of PSGL-1. For example the first polypeptide less than 400 amino acids in length, e.g., less than or equal to 300, 250, 150, 100, 50, or 25 amino acids in length. Exemplary PSGL-1 polypeptide and nucleic acid sequences include GenBank Access No: A57468; XP006867; XM006867; XP140694 and XM140694.

The tumor vaccine fusion protein also includes a third other polypeptides, for example an immunoglobulin polypeptide or at least a region of an immunoglobulin polypeptide. “At least a region” is meant to include any portion of an immunoglobulin molecule, such as the light chain, heavy chain, FC region, Fab region, Fv region or any fragment thereof. Immunoglobulin fusion polypeptide are known in the art and are described in e.g., U.S. Pat. Nos. 5,516,964; 5,225,538; 5,428,130; 5,514,582; 5,714,147; and 5,455,165.

The immunoglobulin polypeptide comprises a full-length immunoglobulin polypeptide. Alternatively, the immunoglobulin polypeptide comprise less than full-length immunoglobulin polypeptide, e.g., a heavy chain, light chain, Fab, Fab₂, Fv, or Fc. Preferably, the immunoglobulin polypeptide includes the heavy chain of an immunoglobulin polypeptide. More preferably the immunoglobulin polypeptide includes the Fc region of an immunoglobulin polypeptide.

In another aspect of the invention the immunoglobulin polypeptide has less effector function that the effector function of a Fc region of a wild-type immunoglobulin heavy chain. Fc effector function includes for example, Fc receptor binding, complement fixation and T cell depleting activity. (see for example, U.S. Pat. No. 6,136,310) Methods of assaying T cell depleting activity, Fc effector function, and antibody stability are known in the art. In one embodiment the second polypeptide has low or no affinity for the Fc receptor. In an alternative embodiment, the immunoglobulin polypeptide has low or no affinity for complement protein C1q.

Expression of Mucin-IRS Fusion Protein-Containing Vaccines

Another aspect of the invention pertains to vectors, preferably expression vectors, containing a nucleic acid encoding mucin polypeptides, or derivatives, fragments, analogs or homologs thereof. In various aspects the vector contains a nucleic acid encoding a mucin polypeptide operably linked to an nucleic acid encoding an IRS polypeptide, or derivatives, fragments analogs or homologs thereof. Additionally, the vector comprises a nucleic acid encoding an α1,2 fucosyltransferase, an α1,3 fucosyltransferase or similar enzyme. As used herein, the term “vector” refers to a nucleic acid molecule capable of transporting another nucleic acid to which it has been linked. One type of vector is a “plasmid”, which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention are introduced into host cells to thereby produce proteins or peptides, including fusion proteins or peptides, encoded by nucleic acids as described herein (e.g., mucin-IRS fusion polypeptides, mutant forms of mucin-IRS fusion polypeptides, etc.).

The recombinant expression vectors of the invention are designed for expression of mucin-IRS fusion polypeptides in prokaryotic or eukaryotic cells. For example, vaccines containing mucin-IRS fusion polypeptides are expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector is transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11 d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention is carried out by standard DNA synthesis techniques.

In another embodiment, the mucin-IRS fusion polypeptide expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, mucin-IRS fusion polypeptides are expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell is any prokaryotic or eukaryotic cell. For example, fusion polypeptides are expressed in bacterial cells such as E. coli, insect cells, yeast or mammalian cells (such as human, Chinese hamster ovary cells (CHO) or COS cells). Other suitable host cells are known to those skilled in the art.

Vector DNA is introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells are found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker is introduced into a host cell on the same vector as that encoding the vaccines containing mucin fusion polypeptides, or are introduced on a separate vector. Cells stably transfected with the introduced nucleic acid are identified by drug selection (e.g., cells that have incorporated the selectable marker gene will survive, while the other cells die).

A host cell of the invention, such as a prokaryotic or eukaryotic host cell in culture, is used to produce (i.e., express) mucin-IRS fusion polypeptides. Accordingly, the invention further provides methods for producing mucin-IRS fusion polypeptides using the host cells of the invention. In one embodiment, the method comprises culturing the host cell of invention (into which a recombinant expression vector encoding mucin-IRS fusion polypeptides has been introduced) in a suitable medium such that mucin-IRS fusion polypeptides is produced. In another embodiment, the method further comprises isolating mucin-IRS polypeptide from the medium or the host cell.

The vaccines containing mucin-IRS fusion polypeptides are isolated and purified in accordance with conventional conditions, such as extraction, precipitation, chromatography, affinity chromatography, electrophoresis or the like. For example, the vaccines are purified by passing a solution through a column which contains immobilized protein A or protein G which selectively binds the Fc portion of the fusion protein. See, for example, Reis, K. J., et al., J. Immunol. 132:3098-3102 (1984); PCT Application, Publication No. WO87/00329. The fusion polypeptide may the be eluted by treatment with a chaotropic salt or by elution with aqueous acetic acid (1 M).

Alternatively, an mucin-IRS fusion polypeptides according to the invention are chemically synthesized using methods known in the art. Chemical synthesis of polypeptides is described in, e.g., A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The polypeptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of polypeptides facilitates the incorporation of modified or unnatural amino acids, including D-amino acids and other small organic molecules. Replacement of one or more L-amino acids in a peptide with the corresponding D-amino acid isoforms is used to increase the resistance of peptides to enzymatic hydrolysis, and to enhance one or more properties of biologically active peptides, i.e., receptor binding, functional potency or duration of action. See, e.g., Doherty, et al., 1993. J. Med. Chem. 36: 2585-2594; Kirby, et al., 1993. J. Med. Chem. 36:3802-3808; Morita, et al., 1994. FEBS Lett. 353: 84-88; Wang, et al., 1993. Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the polypeptide backbone. This strategy is used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus (e.g., with K₃Fe(CN)₆ at pH 8.5) (Samson et al., Endocrinology, 137: 5182-5185 (1996)), or between two amino acid side chains. See, e.g., DeGrado, Adv Protein Chem, 39: 51-124 (1988). Disulfide bridges are also introduced into linear sequences to reduce their flexibility. See, e.g., Rose, et al., Adv Protein Chem, 37: 1-109 (1985); Mosberg et al., Biochem Biophys Res Commun, 106: 505-512 (1982). Furthermore, the replacement of cysteine residues with penicillamine (Pen, 3-mercapto-(D) valine) has been used to increase the selectivity of some opioid-receptor interactions. Lipkowski and Carr, Peptides: Synthesis, Structures, and Applications, Gutte, ed., Academic Press pp. 287-320 (1995).

Pharmaceutical Compositions

The vaccines and fusion peptides and nucleic acids of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements, which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. a vaccine or fusion protein, in a vector by expression from encoding DNA in a viral vector (see for example, EP-A-415731 and WO 90/07936).

In a specific embodiment of the present invention, nucleic acids include a sequence that encodes a vaccine, or functional derivatives thereof, are administered to modulate immune cell activation by way of gene therapy. In more specific embodiments, a nucleic acid or nucleic acids encoding a vaccine or fusion protein, or functional derivatives thereof, are administered by way of gene therapy. Gene therapy refers to therapy that is performed by the administration of a specific nucleic acid to a subject. In this embodiment of the present invention, the nucleic acid produces its encoded peptide(s), which then serve to exert a therapeutic effect by modulating function of the disease or disorder. Any of the methodologies relating to gene therapy available within the art may be used in the practice of the present invention. See e.g., Goldspiel, et al., 1993. Clin Pharm 12: 488-505.

In a preferred embodiment, the Therapeutic comprises a nucleic acid that is part of an expression vector expressing any one or more of the vaccines, fusion proteins, or fragments, derivatives or analogs thereof, within a suitable host. In a specific embodiment, such a nucleic acid possesses a promoter that is operably-linked to coding region(s) of a fusion protein. The promoter may be inducible or constitutive, and, optionally, tissue-specific. In another specific embodiment, a nucleic acid molecule is used in which coding sequences (and any other desired sequences) are flanked by regions that promote homologous recombination at a desired site within the genome, thus providing for intra-chromosomal expression of nucleic acids. See e.g., Koller and Smithies, 1989. Proc Natl Acad Sci USA 86: 8932-8935.

Delivery of the Therapeutic nucleic acid into a patient may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. In a specific embodiment of the present invention, a nucleic acid is directly administered in vivo, where it is expressed to produce the encoded product. This may be accomplished by any of numerous methods known in the art including, e.g., constructing the nucleic acid as part of an appropriate nucleic acid expression vector and administering the same in a manner such that it becomes intracellular (e.g., by infection using a defective or attenuated retroviral or other viral vector; see U.S. Pat. No. 4,980,286); directly injecting naked DNA; using microparticle bombardment (e.g., a “Gene Gun®; Biolistic, DuPont); coating the nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis (see, e.g., Wu and Wu, 1987. J Biol Chem 262: 4429-4432), which can be used to “target” cell types that specifically express the receptors of interest, etc.

An additional approach to gene therapy in the practice of the present invention involves transferring a gene into cells in in vitro tissue culture by such methods as electroporation, lipofection, calcium phosphate-mediated transfection, viral infection, or the like. Generally, the method of transfer includes the concomitant transfer of a selectable marker to the cells. The cells are then placed under selection pressure (e.g., antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including, e.g., transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. See e.g., Loeffler and Behr, 1993. Meth Enzymol 217: 599-618. The chosen technique should provide for the stable transfer of the nucleic acid to the cell, such that the nucleic acid is expressible by the cell. Preferably, the transferred nucleic acid is heritable and expressible by the cell progeny.

In preferred embodiments of the present invention, the resulting recombinant cells may be delivered to a patient by various methods known within the art including, e.g., injection of epithelial cells (e.g., subcutaneously), application of recombinant skin cells as a skin graft onto the patient, and intravenous injection of recombinant blood cells (e.g., hematopoietic stem or progenitor cells). The total amount of cells that are envisioned for use depend upon the desired effect, patient state, and the like, and may be determined by one skilled within the art.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (International Patent Publication WO 94/08598), neural stem cells (Stemple and Anderson, 1992, Cell 71: 973-985), hematopoietic stem or progenitor cells, e.g., as obtained from bone marrow, umbilical cord blood, peripheral blood, fetal liver, and the like. In a preferred embodiment, the cells utilized for gene therapy are autologous to the patient.

The vaccines of the present invention also include one or more adjuvant compounds. Adjuvant compounds are useful in that they enhance long term release of the vaccine by functioning as a depot. Long term exposure to the vaccine should increase the length of time the immune system is presented with the antigen for processing as well as the duration of the antibody response. The adjuvant compound also interacts with immune cells, e.g., by stimulating or modulating immune cells. Further, the adjuvant compound enhances macrophage phagocytosis after binding the vaccine as a particulate (a carrier/vehicle function).

Adjuvant compounds useful in the present invnetion include Complete Freund's Adjuvant (CFA); Incomplete Freund's Adjuvant (IFA); Montanide ISA (incomplete seppic adjuvant); Ribi Adjuvant System (RAS); TiterMax; Syntex Adjuvant Formulation (SAF); Aluminum Salt Adjuvants; Nitrocellulose-adsorbed antigen; Encapsulated or entrapped antigens; Immune-stimulating complexes (ISCOMs); and Gerbu^{R} adjuvant.

Methods of Immunization

The invention provides a method of immunization of a subject. A subject is immunized by administration to the subject the vaccine of the invention. The subject is a mammal, such as a human, at risk of developing or suffering from cancer. Cancer includes for example, breast, lung, colon, prostate, pancreatic, cervical cancer and melanoma). The cancer carry the the Le^{y} epitope.

The cell surface of cancer cells often contains specific carbohydrates, polypeptides and other potential antibody epitopes that are not presence on the surface of non-cancerous cells. This antigen disparity allows the body's immune system to detect and respond to cancer cells. Mucin polypeptides have been associated with numerous cancers. For example, PSGL-1 has been associated with cancers, including lung cancer and acute myeloid leukemia (See Kappelmayer et al., Br J Haematol. 2001, 115(4):903-9). Also, MUC1-specific antibodies have been detected in sera from breast, pancreatic and colon cancer patients. It is clear that mucins are recognized by the human immune system; therefore, immunity against tumor cells expressing specific antigens will be induced by vaccines containing mucin-IRS fusion proteins and a tumor cell-specific antigen. Immunity to tumor cells is measured by the extent of decrease of tumor size, decreased tumor vascularization, increased subject survival, or increased tumor cell apoptosis.

The vaccines of the present invention possess superior immunoprotective and immunotherapeutic properties over other vaccine lacking adjuvant polypeptides. Mucin-IRS fusion protein-containing vaccines have enhanced immunogenicity compared to vaccines to tumor antigens known in the art, safety, tolerability and efficacy. For example, the enhanced immunogenicity of the vaccine of the present invention is greater than comparative non-adjuvant polypeptide-containing vaccines by 1.5-fold, 2-fold, 3-fold, 5-fold, 10-fold, 20-fold, 50-fold, 100-fold or more, as measured by stimulation of an immune response such as antibody production and/or secretion, activation and expansion of T cells, and cytokine expression (e.g., production of interleukins).

The methods described herein lead to a reduction in the severity or the alleviation of one or more symptoms of a cancer. Cancers diagnosed and or monitored, typically by a physician using standard methodologies For example cancer is diagnosed by physical exam, biopsy, blood test, or x-ray.

The subject is e.g., any mammal, e.g., a human, a primate, mouse, rat, dog, cat, cow, horse, pig. The treatment is administered prophylactically. Alternatively, treatment is administered therapeutically.

Efficaciousness of treatment is determined in association with any known method for diagnosing or treating the particular disorder Alleviation of one or more symptoms of the disorder indicates that the compound confers a clinical benefit. By “efficacious” is meant that the treatment leads to decrease in size, prevalence, or metastatic potential of the cancer in a subject. When treatment is applied prophylactically, “efficacious” means that the treatment retards or prevents a tumor from forming or retards, prevents, or alleviates a symptom the cancer. Assessment of cancer is made using standard clinical protocols.

Methods of Increasing Immune Cell Activation

The invention provides a method of activating or stimulating an immune cell (e.g., a B cell or a T cell). T cell activation is defined by an increase in calcium mediated intracellular cGMP, or an increase in cell surface receptors for IL-2. For example, an increase in T cell activation is characterized by an increase of calcium mediated intracellular cGMP and or IL-2 receptors following contacting the T cell with the vaccine, compared to in the absence of the vaccine. Intracellular cGMP is measured, for example, by a competitive immunoassay or scintillation proximity assay using commercially available test kits. Cell surface IL-2 receptors are measured, for example, by determining binding to an IL-2 receptor antibody such as the PC61 antibody. Immune cell activation can also be determined by measuring B cell proliferative activity, polyclonal immunoglobulin (Ig) production, and antigen-specific antibody formation by methods known in the art.

The invention will be further illustrated in the following non-limiting examples.

### EXAMPLE 1

### Production of Adjuvant Polypeptides

Transfections and Production of Secreted PSGL-1/mIgG_{2b} Chimeras

The transfection cocktailare prepared by mixing 39 μl of 20% glucose, 39 μg of plasmid DNA, 127 μl dH₂O, and 15.2 μl 0.1M polyethylenimine (25 kDa; Aldrich, Milwaukee, Wis.) in 5-ml polystyrene tubes. In all transfection mixtures, 13 μg of the PSGL-1/mIgG_{2b} plasmid are used. Thirteen micrograms of the plasmid for the different fucosyltransferases were added, and, when necessary, the CDM8 plasmid was added to reach a total of 39 μg of plasmid DNA. The mixtures were left in room temperature for 10 min before being added in 10 ml of culture medium to the cells, at approximately 70% confluency. After 7 days, cell supernatants were collected, debris spun down (1400×g, 15 mm) and NaN₃ was added to a final concentration of 0.02% (w/v).

Purification of Secreted PSGL-1/mIgG_{2b}, for SDS-PAGE and Western Blot Analysis

PSGL-1/mIgG_{2b} fusion proteins are purified from collected supernatants on 50 μl goat anti-mIgG agarose beads (100:1 slurry; Sigma) by rolling head over tail overnight at 4° C. The beads with fusion proteins are washed three times in PBS and used for subsequent analysis. Typically, the sample was dissolved in 50 μl of 2× reducing sample buffer and 10:1 of sample was loaded in each well.

ELISA for Determination of PSGL-1/mIgG_{2b} Concentration in Supernatants

Ninety-six-well ELISA plates (Costar 3590, Corning, N.Y.) are coated with 0.5 μg/well of affinity-purified goat anti-mIgG specific antibodies (Sigma) in 50 μl of 50 mM carbonate buffer, pH 9.6, for two h in room temperature. After blocking o/n at 4° C. with 300 μl 3% bovine serum albumin (BSA) in PBS with 0.05% Tween (PBS-T) and subsequent washing, 50 μl sample supernatant is added, serially diluted in culture medium. Following washing, the plates are incubated for 2 h with 50 μl of goat anti-mIgM-HRP (Sigma), diluted 1:10,000 in blocking buffer. For the development solution, one tablet of 3,3′,5,5′-tetramethylbenzidine (Sigma) was dissolved in 11 ml of 0.05 M citrate/phosphate buffer with 3 μl 30% (w/v) H₂O₂. One hundred microliters of development solution is added. The reaction is stopped with 25 μl 2 M H₂SO₄. The plates are read at 450 and 540 nm in an automated microplate reader (Bio-Tek Instruments, Winooski, Vt.). As a standard, a dilution series of purified mIgG Fe fragments (Sigma) in culture medium is used in triplicate.

SDS-PAGE and Western Blotting

SDS-PAGE is run by the method of Laemmli (1970) with a 5% stacking gel and an 8% resolving gel, and separated proteins are electrophoretically blotted onto Hybond™-C extra membranes as described before (Liu et al., 1997). Following blocking overnight in Tris-buffered saline with 0.05% Tween-20 (TBS-T) with 3% BSA, the membranes are washed three times with TBS-T. They are then incubated for 1 h in room temperature with antobody. All antibodies are diluted 1:200 in 3% BSA in TBS-T. The membranes are washed three times with TBS-T before incubation for 1 h at room temperature with secondary horseradish peroxidase (HRP)-conjugated antibodies, goat anti-mIgM (Cappel, Durham, N.C.) or goat anti-mIgG₃ (Serotec, Oxford, England) diluted 1:2000 in 3% BSA in TBS-T. Bound secondary antibodies are visualized by chemiluminescence using the ECL kit (Amersham Pharmacia Biotech, Uppsala, Sweden) according to the instructions of the manufacturer. For detection of the PSGL-1/mIgG_{2b} itself, HRP-labeled goat anti-mIgG (Sigma) is used at a dilution of 1:10,000 in 3% BSA in TBS-T as described, but without incubation with a secondary antibody.

### EXAMPLE 2

### Production of Lewis Y-PSGL-1/mIg-T Cell Stimulator-Conjugated Vaccines

The data described herein was generated using the following reagents and methods.

Cell culture: COS-7 m6 cells (Seed, 1987), CHO-K1 (ATCC CCL-61), and the SV40 Large T antigen expressing 293 human embryonic kidney cell line, are cultured in Dulbecco's modified Eagle's medium (GibcoBrl, Life Technologies, Paisley, Scotland), supplemented with 10% fetal bovine serum (GibcoBrl, Life Technologies), 25 μ/ml gentamycin sulfate (Sigma, St. Louis, Mo.) and 2 mM glutamine (GibcoBrl, Life Technologies). The cells are passaged every 2-4 days. The HH14 hybndoma(ATCC HB-9299; U.S. Pat. No. 4,857,639) are cultured in RPMI 1640 (GibcoBrl, Life Technologies), supplemented with 10% fetal bovine serum, 100 U/ml of penicillin, 100 μg/μl of streptomycin, and 2 mM glutamine.

Materials. Crosslinker N-[γ-maleimidobutyryloxy]sulfosuccinnimide ester (Sulfo-GMBS) (22324, PIERCE, Rockford. Ill. 61105). Coupling buffer: 20 mM sodium phosphate, 0.15 M NaCl, 0.1 M EDTA, pH 7.2 Hi Trap™ Desalting column (17-1408-01, Amersham Biosciences, SE-75184 Uppsala, Sweden). HiPrep™ 16/60 Sephacryl™ S-200 column(17-1166-01, Amersham Biosciences, SE-75184 Uppsala, Sweden).

Vaccine production methods. The Le^{y} substituted PSGL1/mIgG_{2b} is resuspended in coupling buffer to a concentration of 2 mg/ml. 200 μl of resuspended Le^{y} substituted PSGL1/mIgG_{2b} was transferred to a 10 ml tube. 2 mg of Sulfo-GMBS is dissolved in 1 ml of conjugation buffer, and 100 μl of the Sulfo-GMBS solution was immediately transferred into the test tube containing the Le^{y} substituted PSGL1/mIgG_{2b}. Incubate for 2 hours in room temperature. Equilibrate the desalting column with 15 ml of coupling buffer. Apply the 300 μl of reaction solution onto the Hi Trap™ desalting column using an FPLC system. Elute with 0.5 ml aliquots of coupling buffer. Monitor the eluted protein by absorbance at 280 nm. The maleimide-activated Le^{y} substituted PSGL1/mIgG_{2b} should elute in fraction 5-6. Dissolve an appropriate amount of a T cell stimulator (keyhole limpet hemocyanin, a heat shock protein, or a superantigen) in 500 μl of coupling buffer overnight at room temperature. Add T cell stimulator solution to pooled fractions containing maleimide-activated Le^{y} substituted PSGL1/mIgG_{2b}. Incubate for 3 hours at room temperature. Add 8 M guanidine solution to the test tube containing the conjugated T cell stimulator-PSGL-1/mIgG_{2b} protein (the “coupling protein”), until the concentration of guanidine reach 6 M. Equilibrate the HiPrep™ 16/60 Sephacryl™ S-200 column with 100 ml of PBS. Apply the 4.5 ml of reaction volume onto the column run in the FPLC system. Elute with 1.0 ml aliquots of PBS. Monitor the protein elution by absorbance at 280 nm. The coupling protein should elute in fraction 35-38 (see FIGS. 6 and 7). Dialyze against water to remove PBS. Freeze and lyophilize the coupling protein. Characterize the coupling protein by ELISA and Western blot analysis.

Vaccine Testing Methods.

Mice are immunized by intraperitoneal injection with the Le^{y} substituted mucin/Ig alone or conjugated to the an IRS polypeptide (e.g., keyhole limpet hemocyanin, an heat shoch protein, or a superantigen). Control vaccines are mucin/Igs, either alone or conjugated to the IRS polypeptides, which are not Le^{y} substituted, or other proteins carrying monovalent or oligovalent Le^{y} substitution. The multivalent expression of Le^{y} on the mucin/Ig facilitates a more efficient B cell stimulation than the other Le^{y}-proteins. The humoral response to Le^{y} results after primary, secondary and tertiary immunizations. Spleen cells from mice immunized three times are collected and restimulated ex vivo. Cell division (as measured by thymidine incorporation or other method known to one skilled in the art), and cytokine production (ELIspot) are measured. The CD4/CD8 phenotype of the responding cells is also assessed.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A purified tumor vaccine comprising a polypeptide wherein the polypeptide is glycosylated by a α1,2 fucosyltransferase and α1,3 fucosyltransferase.

2. The tumor vaccine of claim 1, wherein the polypeptide comprises multiple Le^{y} epitopes.

3. The tumor vaccine of claim 1, wherein the polypeptide comprises at least a region of a P-selectin glycoprotein ligand-1.

4. The tumor vaccine of claim 1, wherein the polypeptide includes an extracellular portion of a P-selectin glycoprotein ligand-1.

5. The tumor vaccine of claim 3 or 4, wherein the polypeptide comprises more Le^{y} epitopes epitopes than a wild-type P-selectin glycoprotein ligand-1 polypeptide.

6. The tumor vaccine of claim 1, wherein the polypeptide is a dimer.

7. A purified tumor vaccine comprising, a first polypeptide operably linked to a second polypeptide, wherein the first polypeptide comprises a tumor-associated carbohydrate epitope and the second polypeptide comprises an immune response stimulator polypeptide.

8. The tumor vaccine of claim 7, wherein the tumor-associated carbohydrate epitope is selected from the group consisting of Lewis A, Lewis X, Lewis Y, and Lewis B.

9. The tumor vaccine of claim 7, wherein the first polypeptide comprises mutiple tumor-associated carbohydrate eptiopes.

10. A purified tumor vaccine comprising a first polypeptide operably linked to a second polypeptide, wherein the first polypeptide is glycosylated by an α1,2 fucosyltransferase and α1,3 fucosyltransferase and the second polypeptide comprises an immune response stimulator polypeptide.

11. The tumor vaccine of claim 7 or 10, wherein the immune response stimulator polypeptide is a T-cell stimulator polypeptide.

12. The tumor vaccine of claim 11, wherein the T-cell stimulator polypetide is selected from the group comprising keyhole limpet hemocyanin, a heat shock protein (HSP) and a superantigen.

13. The tumor vaccine of claim 12, wherein the heat shock protein is HSP60 or HSP70.

14. The tumor vaccine of claim 12, wherein the superantigen is Staphylococcus enterotoxin.

15. The tumor vaccine of claims 7 or 10, wherein the first polypeptide comprises multiple Le^{y} epitopes.

16. The tumor vaccine of claim 7 or 10, wherein the first polypeptide comprises at least a region of a P-selectin glycoprotein ligand-1.

17. The tumor vaccine of claims 7 or 10, wherein the first polypeptide includes an extracellular portion of a P-selectin glycoprotein ligand-1.

18. The tumor vaccine of claim 16 or 17, wherein the first polypeptide comprises more Le^{y} epitopes than a wild-type P-selectin glycoprotein ligand-1 polypeptide.

19. The tumor vaccine of claims 7 or 10, further comprising an immunoglobulin polypeptide.

20. The tumor vaccine of claim 19, wherein the immunoglobulin polypeptide comprises a region of a heavy chain immunoglobulin polypeptide.

21. The tumor vaccine of claim 19, wherein the immunoglobulin polypeptide comprises an Fc region of an immunoglobulin heavy chain.

22. The tumor vaccine of claims 7 or 10, wherein the vaccine is a dimer.

23. A purified tumor vaccine comprising:
(a) an adjuvant polypeptide comprising a first polypeptide operably linked to a second polypeptide, wherein the first polypeptide is a mucin polypeptide and is glycosylated by a α1,2 fucosyltransferase and α1,3 fucosyltransferase and the second polypeptide comprises at least a region of an immunoglobulin polypeptide and
(b) an immune response stimulator polypeptide.

24. The tumor vaccine of claim 23, wherein the adjuvant polypeptide is operably linked to the immune response stimulator polypeptide.

25. The tumor vaccine of claim 23, wherein the adjuvant polypeptide is covalently linked to the immune response stimulator polypeptide.

26. The tumor vaccine of claim 23, wherein the first polypeptide comprises at least a region of a P-selectin glycoprotein ligand-1.

27. The tumor vaccine of claim 23, wherein the first polypeptide includes an extracellular portion of a P-selectin glycoprotein ligand-1.

28. The tumor vaccine of claim 23, wherein the first polypeptide comprises multiple Le^{y} epitopes.

29. The vaccine of claims 25 or 26, wherein the first polypeptide comprises more Le^{y} epitopes epitopes than a wild-type P-selectin glycoprotein ligand-1 polypeptide.

30. The tumor vaccine of claim 23, wherein the second polypeptide comprises a region of a heavy chain immunoglobulin polypeptide.

31. The vaccine of claim 25, wherein said second polypeptide comprises an Fe region of an immunoglobulin heavy chain.

32. The vaccine of claim 25, wherein the adjuvant polypeptide is a dimer.

33. The tumor vaccine of claim 25, wherein the immune response stimulator polypeptide a T-cell stimulator polypeptide.

34. The tumor vaccine of claim 33, wherein the T-cell stimulator polypetide is selected from the group comprising keyhole limpet hemocyanin, a heat shock protein (HSP) and a superantigen.

35. The tumor vaccine of claim 34, wherein the heat shock protein is HSP60 or HSP70.

36. The tumor vaccine of claim 34, wherein the superantigen is Staphylococcus enterotoxin.

37. An isolated nucleic acid encoding the vaccine of any one of claims 1, 7, 10 or 24.

38. A vector comprising the nucleic acid of claim 37.

39. A cell comprising the vector of claim 38.

40. A method immunization in a subject in need thereof, the method comprising administering to the subject the tumor vaccine of claim 1.

41. A method of treating cancer in a subject in need thereof, the method comprising administering to the subject the tumor vaccine of claim 1.

42. The tumor vaccine of claim 1, wherein the α1,2 fucosyltransferase is FUT1 or FUT2.

43. The tumor vaccine of claim 1, wherein the α1,3 fucosyltransferase is selected from the group consisting of FUT3, FUT4, FUT5, FUT6, FUT7, or FUT9.

44. The tumor vaccine of claim 1, wherein the α1,2 fucosyltransferase is FUT1 and the α1,3 fucosyltransferase is selected from the group consisting of FUT4, FUT5, or FUT6.

45. A method of increasing immune cell activation, comprising contacting said immune cell with a purified tumor vaccine, said tumor vaccine comprising a polypeptide, wherein the polypeptide is glycosylated by a α1,2 fucosyltransferase and α1,3 fucosyltransferase, such that the activation of said cell is increased.

46. The method of claim 45, wherein said cell is selected from the group consisting of a B-cell and a T-cell.
